(19) United States
(12) Patent Application Publication
(10) Pub. No.: US 2018/0012283 A1
(43) Pub. Date: Jan. 11, 2018

(54) SENSOR BASED PRODUCT RECOMMENDATIONS

(71) Applicant: eBay Inc., San Jose, CA (US)

(72) Inventors: Subrahmanya Harve, San Jose, CA (US); Deepak Kumar Vasthimal, San Jose, CA (US)

(21) Appl. No.: 15/203,360

(22) Filed: Jul. 6, 2016

(51) Int. Cl. G06Q 30/06; A61B 5/11; A61B 5/00

(52) U.S. Cl. CPC G06Q 30/0631; A61B 5/742; A61B 5/1118; A61B 2503/12; A61B 2562/0219; A61B 2562/0257; A61B 2560/0242

(57) ABSTRACT

A system, method, and computer program product for generating item recommendations based on sensor data captured by sensors on client machines. A server processes collected sensor data to estimate user physical activity based on sensor data patterns, and forms a user profile based on the estimated user physical activity. The server associates the user profile with relevant product or service recommendations, based on previous purchases by a user or by others matching the user profile. The user may make a purchase based on the displayed recommendations or forward the recommendations to others. The recommendations include discounts based on the user profile. Subsequent sensor data showing satisfaction with a purchased item serves as a testimonial to user satisfaction with the item. The sensor data may be collected from a user in an augmented or virtual reality system, and the recommendations may be displayed in such a system.

### TECHNICAL FIELD

The present disclosure relates to collecting sensor data from user devices, such as smartphones and fitness monitors, and generating product recommendations from the collected sensor data.

### BACKGROUND

As the use of network-based publication systems and marketplaces such as online commerce services or auction services expands, and the volume of item listings in such applications increases, the speed, ease, and convenience with which product information that is relevant to customers may be retrieved from such marketplaces increases in importance to customers.

### BRIEF DESCRIPTION OF DRAWINGS

Some embodiments are illustrated by way of example and not limitation in the figures of the accompanying drawings in which:

FIG. 1 is a block diagram illustrating a publication system in the example form of a network-based marketplace system.

FIG. 2 is a diagrammatic representation of marketplace and payment applications.

FIG. 3 is a diagrammatic representation of different sensor data collected by a client machine, in accordance with a disclosed embodiment.

FIG. 4 is a diagrammatic representation of a system for processing sensor data collected by a client machine into relevant recommendations, in accordance with a disclosed embodiment.

FIG. 5 is a flow chart illustrating a method for processing sensor data collected by a client machine into relevant recommendations, in accordance with a disclosed embodiment.

FIG. 6 is a flow chart illustrating a method for processing sensor data collected by multiple client machines into relevant collaborative recommendations, according to an embodiment.

FIG. 7 is a block diagram of a machine in the example form of a computer system within which a set of instructions for causing the machine to perform any one or more of the methodologies discussed herein, may be executed.

FIG. 8 is a diagrammatic view of a data structure according, to an example embodiment of a network-based marketplace.

### DETAILED DESCRIPTION

In the following description, for purposes of explanation, numerous specific details are set forth in order to provide a thorough understanding of some example embodiments. It will be evident, however, to one skilled in the art that the present embodiments may be practiced without these specific details. Examples merely typify possible variations. Unless explicitly stated otherwise, components and functions are optional and may vary in sequence or be combined or subdivided.

Users of networked publication and ecommerce systems often experience difficulty in finding items of interest. The present inventors have realized, among other things, that a user's lifestyle may involve specific needs or problems, so sensor data that captures details of a user's lifestyle may help associate user activities with products and services of interest to the user. Further, other users that share a similar lifestyle may have a wealth of knowledge and a purchase history of products and services that could be leveraged to help a given user get relevant product and service recommendations.

Additionally, once a user has bought a recommended product or service, the sensor data may also provide useful insight regarding the effectiveness of a given product or service for that buyer to potential buyers sharing a similar lifestyle prior to purchase. Thus, rather than relying only on direct reviews of a product or service provided by previous buyers, actual data supporting the satisfaction resulting from a purchase could be provided to the networked publication and ecommerce system.

Further, sellers would appreciate the ability to find buyers that are interested in a given product or service, and the possibility to increase buying opportunities and to encourage collaboration between buyers. This increased interaction benefits both the shopper, who may have a better chance of finding what he or she wants or needs, and the ecommerce site, which may experience higher sales. It is therefore useful and helpful to suggest relevant products and services to a shopper.

The system, method, and computer program product disclosed herein thus may provide relevant sensor based product and service recommendations for shoppers on networked publishing and ecommerce sites. Accordingly, one or more of the methodologies discussed herein may obviate a need for additional searching or navigation by the user, which may have the technical effect of reducing computing resources used by one or more devices within the system. Examples of such computing resources include, without limitation, processor cycles, network traffic, memory usage, storage space, and power consumption.

In one example, a method for generating product recommendations may include collecting sensor data with at least one sensor in at least one client machine, transferring the collected sensor data to a server, and estimating user physical activity with the server based on patterns in the collected sensor data. The server may form a user profile based on the estimated user physical activity, associate the user profile with product recommendations, and display the product recommendations to the user.

In another example, a system may include a number of sensors in a client machine that collects sensor data and transfers it to a server that estimates user physical activity based on patterns in the collected sensor data. The server may form a user profile based on the estimated user physical activity, associate the user with a number of product and/or service recommendations, and display the recommendations to a user via an application.

In a further example, a computer-readable hardware medium may store program instructions that, when executed by one or more processors, may collect sensor data from sensors in a client machine, transfer the collected sensor data to a server, and estimate the physical activity of a user based on patterns in the collected sensor data. The server may form a user profile based on the estimated user physical activity and associate the user profile with recommendations for relevant products and/or services. The server may then display the recommendations to a user for a variety of user actions, including making a purchase based on the recommendations and/or suggesting the recommendations another user.

### Architecture

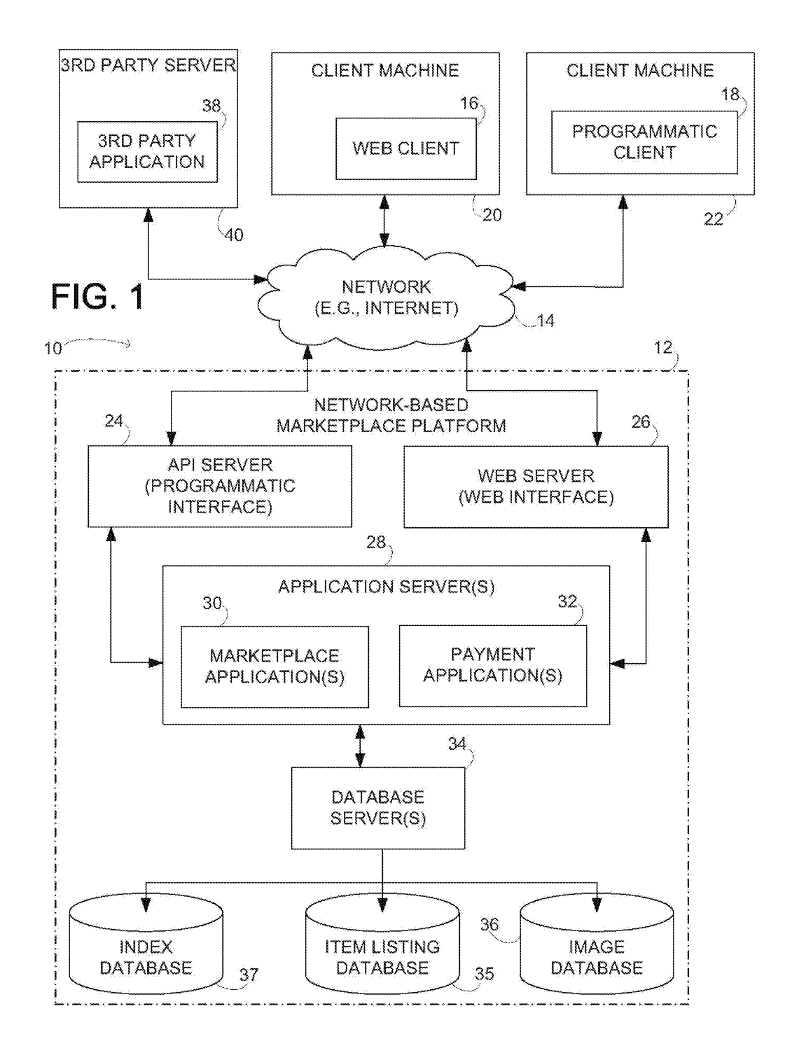

One example of a distributed network implementing a publication system is illustrated in the network diagram of FIG. 1, which depicts a system 10 using a client-server type architecture. A commerce platform, in the example form of a network-based marketplace platform 12, provides server-side functionality, via a network 14 (e.g., the Internet) to one or more clients. As illustrated, the platform 12 interacts with a web client 16 executing on a client machine 20 and a programmatic client 18 executing on a client machine 22. In one embodiment,web client 16 is a web browser, but it may employ other types of web services.

Turning specifically to the exemplary network-based marketplace platform 12, an Application Program Interface (API) server 24 and a web server 26 are coupled to, and provide programmatic and web interfaces respectively to, one or more application servers 28. The application servers 28 may host one or more marketplace applications 30 and payment applications 32. The application servers 28 are, in turn, shown to be coupled to one or more databases servers 34 that may facilitate access to a number of databases, including an item listing database 35, an image database 36, and an index database 37. The item listing database 35 may store data indicative of item listings for items which are offered for sale or auction on the platform 12.

Each item listing may include, inter alia, a text description of the relevant item and metadata categorizing the item. The image database 36 may include images associated with respective item listings in the item listing database 35. The images in the image database 36 may be standard format image files such as Joint Photographic Expert Group (JPEG) files. The index database 37 may contain index data relating to images in the image database to permit image-based searching of the image database 36.

The marketplace applications 30 may provide a number of marketplace functions and services to users that access the marketplace platform 12. The payment applications 32 likewise may provide a number of payment services and functions to users. The payment applications 32 may allow users to quantify, and accumulate, value (e.g., in a commercial currency, such as the U.S. dollar, or a proprietary currency, such as “points”) in accounts, and then to later redeem the accumulated value for products (e.g., goods or services) that are made available via the marketplace applications 30. While the marketplace and payment applications 30 and 32 are shown in FIG. 1 to both form part of the network-based marketplace platform 12, it will be appreciated that, in alternative embodiments, the payment applications 32 may form part of a payment service that is separate and distinct from the marketplace platform 12.

Further, while the system 10 shown in FIG. 1 employs a client-server architecture, the present disclosure is, of course, not limited to such an architecture, and could equally well find application in a distributed, or peer-to-peer, architecture system. The various marketplace and payment applications 30 and 32 could also be implemented as standalone software programs, which do not necessarily have networking capabilities. Additionally, while example embodiments are described with respect to the marketplace platform 12, alternative embodiments may be contemplate use on a publication platform or other non-commerce platforms.

The web client 16, it will be appreciated, may access the various marketplace and payment applications 30 and 32 via the web interface supported by the web server 26. Similarly, the programmatic client 18 may access the various services and functions provided by the marketplace and payment applications 30 and 32 via the programmatic interface provided by the API server 24. The programmatic client 18 may, for example, be a seller application (e.g., the TurboLister application developed by eBay Inc., of San Jose, Calif.) to enable sellers to author and manage listings on the marketplace platform 12 in an off-line manner, and to perform batch-mode communications between the programmatic client 18 and the network-based marketplace platform 12.

FIG. 1 also illustrates a third party application 38, executing on a third party server machine 40, as having programmatic access to the network-based marketplace via the programmatic interface provided by the API server 24. For example, the third party application 38 may, utilizing information retrieved from the network-based marketplace platform 12, support one or more features or functions on a website hosted by the third party. The third party website may, for example, provide one or more promotional, marketplace or payment functions that are supported by the relevant applications of the network-based marketplace platform 12.

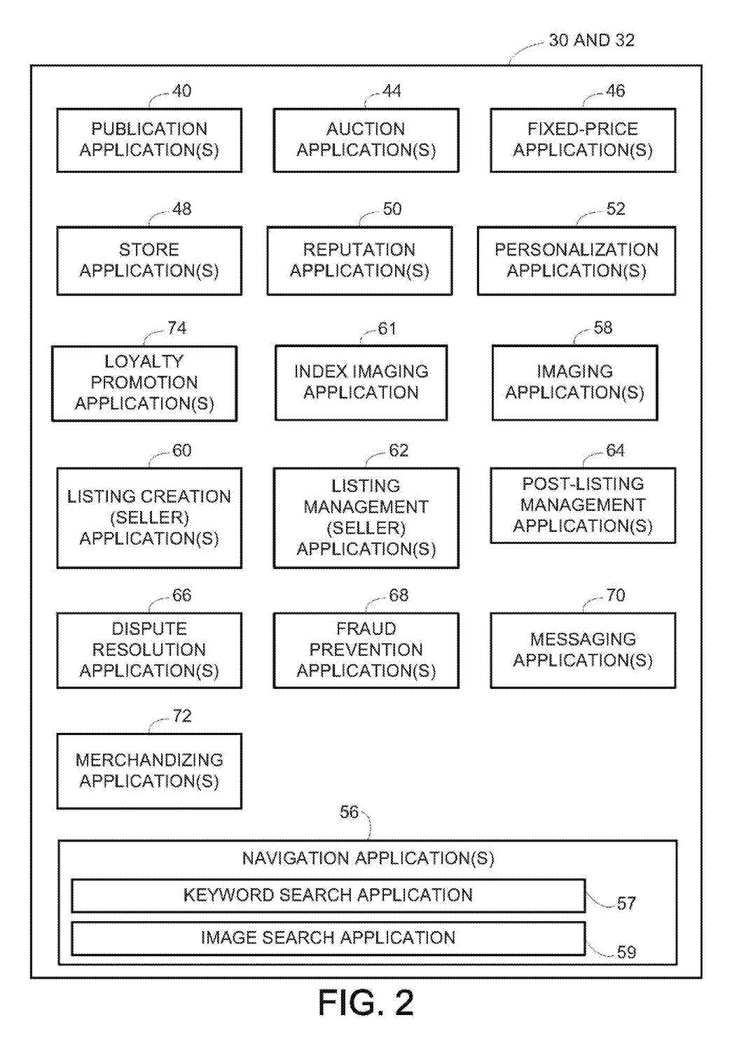

FIG. 2 is a block diagram illustrating multiple marketplace and payment applications 30 and 32 that may be provided as part of the network-based marketplace platform 12. The marketplace platform 12 may provide a number of listing and price-setting mechanisms whereby a seller may list goods or services for sale, a buyer can express interest in or indicate a desire to purchase such goods or services, and a price can be set for a transaction pertaining to the goods or services. To this end, the marketplace applications 30 are shown to include at least one publication application 40 and one or more auction applications 44 which support auction-format listing and price setting mechanisms (e.g., English, Dutch, Vickrey, Chinese, Double, Reverse auctions etc.). The various auction applications 44 may also provide a number of features in support of such auction-format listings, such as a reserve price feature whereby a seller may specify a reserve price in connection with a listing and a proxy bidding feature whereby a bidder may invoke automated proxy bidding.

A number of fixed-price applications 46 may support fixed-price listing formats (e.g., the traditional classified advertisement-type listing or a catalogue listing) and buyout-type listings. Specifically, buyout-type listings (e.g., including the Buy-It-Now (BIN) technology developed by eBay Inc., of San Jose, Calif.) may be offered in conjunction with an auction-format listing, and allow a buyer to purchase goods or services, which are also being offered for sale via an auction, for a fixed-price that is typically higher than the starting price of the auction.

Store applications 48 allow sellers to group their listings within a “virtual” store, which may be branded and otherwise personalized by and for the sellers. Such a virtual store may also offer promotions, incentives, and features that are specific and personalized to a relevant seller.

Reputation applications 50 allow parties that transact utilizing the network-based marketplace platform 12 to establish, build, and maintain reputations, which may be made available and published to potential trading partners. Consider that where, for example, the network-based marketplace platform 12 supports person-to-person trading, users may have no history or other reference information whereby the trustworthiness and credibility of potential trading partners may be assessed. The reputation application 50 allows a user (for example, through feedback provided by other transaction partners) to establish a reputation within the network-based marketplace platform 12 over time. Other potential trading partners may then reference such a reputation for the purposes of assessing credibility and trustworthiness.

Personalization applications 52 allow users of the marketplace platform 12 to personalize various aspects of their interactions with the marketplace platform 12. For example a user may, utilizing an appropriate personalization application 52, create a personalized reference page at which information regarding transactions to which the user is (or has been) a party may be viewed. Further, a personalization application 52 may enable a user to personalize listings and other aspects of their interactions with the marketplace and other parties.

In one embodiment, the network-based marketplace platform 12 may support a number of marketplaces that are customized, for example, for specific geographic regions. A version of the marketplace may be customized for the United Kingdom, whereas another version of the marketplace may be customized for the United States. Each of these versions may operate as an independent marketplace, or may be customized (or internationalized) presentations of a common underlying marketplace.

Navigation of the network based-marketplace may be facilitated by one or more navigation applications 56. For example, a keyword search application 57 enables keyword searches of listings published via the marketplace platform 12. Similarly, an image search application 59 enables an image-based search of item listings published via the marketplace platform 12. To perform an image-based search, a user may submit a query image, whereupon the image search application 59 may compare the query image to images in the image database to produce a result list of item listings based on a similarity ranking between the query image and the images associated with the respective item listings. The similarity ranking may be established by parsing or processing the query image to provide index data, and thereafter comparing the query image's index data to pre-compiled index data for the listing images. A browsing application may allow users to browse various category, catalogue, or inventory data structures according to which listings may be classified within the marketplace platform 12. Various other navigation applications may be provided to supplement the search and browsing applications.

In order to make listings available via the network-based marketplace as visually informative and attractive as possible, as well as to enable image-based searching, the marketplace applications 30 may include one or more imaging applications 58, which users may use to upload images for inclusion within listings. Images thus uploaded are stored in the image database 36, with each image being associatively linked to at least one item listing in the item listing database 35. One of the imaging applications 58 may also operate to incorporate images within viewed listings. The imaging applications 58 may also support one or more promotional features, such as image galleries that are presented to potential buyers. For example, sellers may pay an additional fee to have an image included within a gallery of images for promoted items.

The marketplace platform 12 may also include an image indexing application 61 to parse or process images uploaded via the imaging application 58, as well as to parse or process query images submitted via the image search application 59. Index data is the result of processing images by the image indexing application 61 and is stored in the index database 37.

Listing creation applications 60 may allow sellers to conveniently author listings pertaining to goods or services that they wish to transact via the marketplace platform 12, and listing management applications 62 may allow sellers to manage such listings. Specifically, where a particular seller has authored and/or published a large number of listings, the management of such listings may present a challenge. The listing management applications 62 may provide a number of features (e.g., auto-relisting, inventory level monitors, etc.) to assist the seller in managing such listings. One or more post-listing management applications 64 may also assist sellers with a number of activities that typically occur post-listing. For example, upon completion of an auction facilitated by one or more auction applications 44, a seller may wish to leave feedback regarding a particular buyer. To this end, a post-listing management application 64 may provide an interface to one or more reputation applications 50, so as to allow the seller conveniently to provide feedback regarding multiple buyers to the reputation applications 50.

Dispute resolution applications 66 may provide mechanisms whereby disputes arising between transacting parties may be resolved. For example, the dispute resolution applications 66 may provide guided procedures whereby the parties are guided through a number of steps in an attempt to settle a dispute. In the event that the dispute cannot be settled via the guided procedures, the dispute may be escalated to a third party mediator or arbitrator.

A number of fraud prevention applications 68 implement various fraud detection and prevention mechanisms to reduce the occurrence of fraud within the marketplace. One of the fraud prevention applications 68 may include automatic image comparison, by use of index data produced by the image indexing application 61 and stored in the index database 37. Such image comparison may be used by the fraud prevention application 68 automatically to detect listing images similar to the query image, and to alert a fraud assessor to such image listings, so that the human assessor can examine the identified item listing to determine whether the identified item listing is a fraudulent listing.

Messaging applications 70 may enable the generation and delivery of messages to users of the network-based marketplace platform 12. Such messages may, for example, advise users regarding the status of listings at the marketplace (e.g., providing “outbid” notices to bidders during an auction process or providing promotional and merchandising information to users)

Merchandizing applications 72 may support various merchandizing functions that are made available to sellers to enable sellers to increase sales via the marketplace platform 12. The merchandizing applications 72 also operate the various merchandizing features that may be invoked by sellers and may monitor and track the success of merchandizing strategies employed by sellers.

The present inventors have realized that the marketplace platform 12 described above may be improved if client machines are provided with additional new functionality. For example, a linkage between the sensor data available from various client machines or devices and the networked publication and marketing systems of FIGS. 1-2 may prove advantageous to both buyers and sellers, as previously described. A client machine indeed may control the product and/or service recommendations provided to a potential buyer via the use of sensor data that it collects. If the user provides consent, the user need not even be aware that the client machine is actively determining, through its sensor data, the presented recommendations. In effect, the sensor data describing, for example, user physical activity is transformed into specific item recommendations.

An improved client machine may comprise, for example, a smart phone, a fitness activity tracker, a computer, a watch, a pedometer, or any other device that contains a sensor or can receive data from a sensor. A sensor may comprise, for example, an accelerometer, a Global Positioning System (GPS) device, a camera, a proximity detector, a gyroscope, a scale, a thermometer, or any other measuring device that provides data about a user's physical motion and/or location. Some client machines may include a number of sensors. For example, there are several different sensors in smart phones today, such as an accelerometer, a gyroscope, a camera, and the like. Some client machines may collect and transfer sensor data from other client machines.

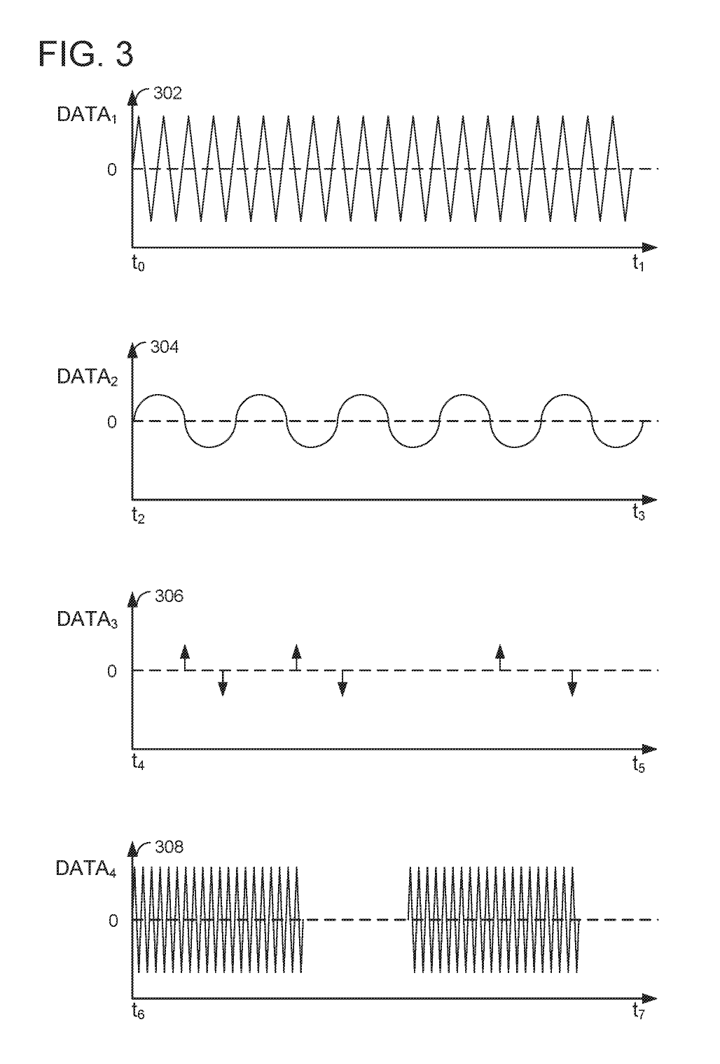

FIG. 3 is a diagrammatic representation of different sensor data collected by a client machine, in accordance with a disclosed embodiment. Sensor data may originate from any number of sensors, and may be shared between different client machines. For purposes of this description, sensor data may actually be measured by one or more sensors contained within one or more client machines, but may still be referred to as being collected by a single client machine. Client machines may comprise, for example, smart phones, fitness tracking devices, and other wearable items containing a sensor.

Client machines may gather sensor data continuously, during predetermined time ranges, or only when a predetermined threshold measured phenomenon level is exceeded, to, for example, maximize battery lifetimes. Data collection may generally involve accessing the various sources of information and observations of user physical activity, and then transporting the data to one or more servers for analysis and storage (e.g., to offload central processing unit (CPU) requirements and to reduce battery usage).

A first example waveform, DATA₁ , shown as item 302, may depict accelerometer data captured when a user is running or jogging. The range of time shown from t₀ to t₁ may comprise several seconds or minutes in this example, though this disclosure is not limited in that respect. The overall time that a user spends running or jogging may extend far beyond the range of time shown here. Accelerometer data for a running or jogging user may be characteristically periodic, with a given frequency range, with sharp transitions that occur such as when a user physically undergoes reversals in at least one direction of motion.

A second example waveform, DATA₂, shown as item 304, may depict accelerometer data captured when a user is walking or bicycling. This waveform may also be periodic, perhaps with lower frequency, but may include smoother directional transitions than those that occur during jogging or running for example. The range of time shown from t₂ to t₃ may again comprise only a subset of the time the user actually spends walking or bicycling.

A third example waveform, DATA₃, shown as item 306, may depict accelerometer data or gyroscope data captured when a user is sleeping. This waveform may be quite sparse as it depicts only those few instances when a user undergoes significant position changes (e.g., “tossing and turning”). The range of time shown from L₄ to t₅ may span eight hours or more, for example, during a typical data gathering session.

A fourth example waveform, DATA₄, shown as item 308, may depict accelerometer data or gyroscope data captured when a user is lifting weights. This waveform may be periodic for a time while the user is moving, followed by a rest period between individual “sets,” for example, followed by one or more further rounds of periodic weight lifting. In this case, the range of time shown from t₆ to t₇ may be tens of seconds to several minutes, though this disclosure is not limited in that respect.

Each of these exemplary and non-limiting waveforms may be sufficiently recognizable to enable reliable detection of a particular physical activity of a user without further data. Generally though, the more data that is available from different types of sensors, the better the overall picture that will be available of a user's physical activity. In some cases, the sensor data may be sufficient to rule out some known user physical activities without detecting exactly what the user is doing.

Examples of user physical activity may include jogging, running, sleeping, driving, walking, lifting weights, bicycling, and remaining still, although this disclosure is not limited, in that respect. The date and time at which a given user physical activity occurs truly also be noted in the sensor data, so that either current or historical user physical activity may be monitored. The sensed user physical activity may also include user activation of the client machine or the triggering of various aspects of its operation (e.g., through taps, key presses, and swipe gestures)

Ambient data from the user's environment (e.g., sound, temperature, or light intensity) may also be provided by sensors. Biometric user data such as the user's skin temperature, galvanic skin response, heat flux, and heart rate may be provided by sensors, such as those in fitness tracking devices. This data may be used to calculate caloric burn, stress level, sleep quality, and other indicators of the user's physical state.

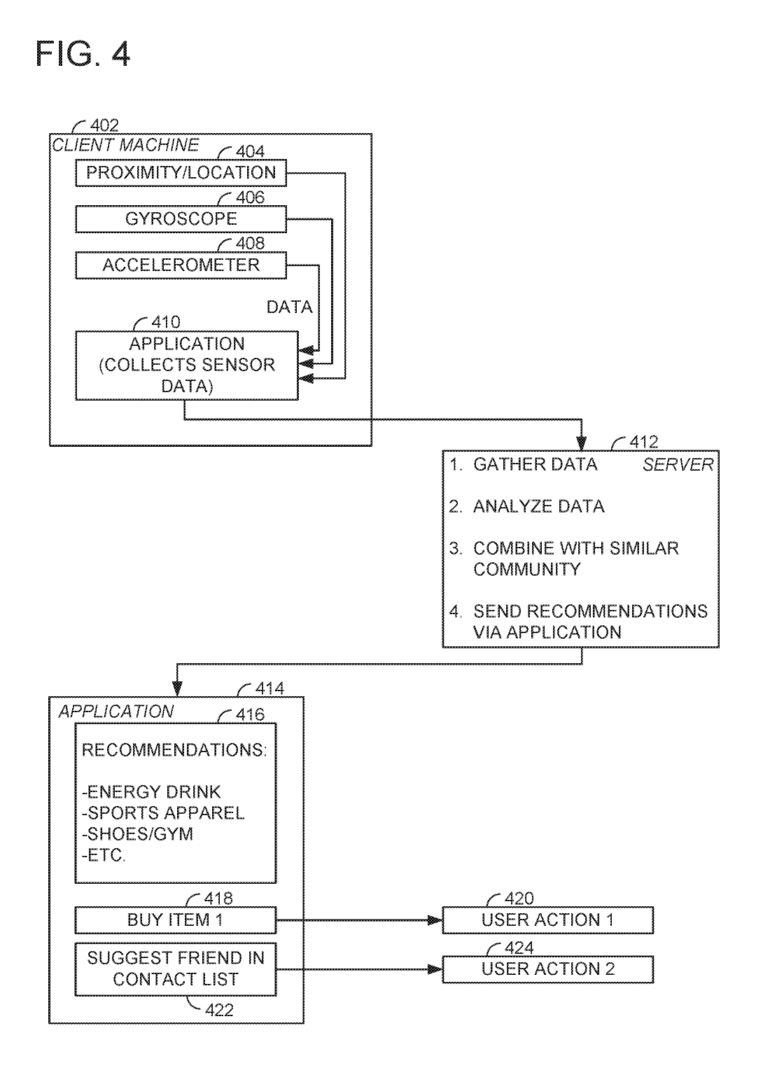

FIG. 4 is a diagrammatic representation of a system for processing sensor data collected by a client machine into relevant recommendations, in accordance with a disclosed embodiment. A client machine 402 may include a proximity/location sensor 404 (which could include a UPS sensor, a cell tower triangulation sensor, or a Wi-Fi based location sensor, for example). The client machine 402 may also include a gyroscope 406, which may determine a spatial orientation. An accelerometer 408 may also be included in the client machine 402 to measure a user's physical accelerations in at least one physical dimension versus time, for example.

The client machine 402 may also include an application 410 that collects sensor data during its execution. The application 410 may reformat and transmit collected sensor data using any known transmission scheme. The application 410 may transmit collected sensor data continuously in substantially real time, or may issue periodic reports summarizing the data, for example, although this disclosure is not limited in that regard.

The client machine 402 may send the collected sensor data to one or more servers 412, or to another client machine, for analysis. The server 412 may generally gather the sensor data and analyze it to determine or at least estimate the user physical activity and location information it represents. The server 412 may have a catalog of sensor data relating to known user physical activities, for example, and pattern recognition tools for matching the collected sensor data to particular physical activities. The client machine 402 may analyse the collected sensor data and send summary data to the one or more servers 412.

Activity types may be determined based on combinations of activity level (e.g., as determined by heart rate), type of movement (e.g., as determined by pattern characteristics, such as waveform frequency and shape), and user location (e.g., as determined by a UPS component of the client machine 402). The server 412 may process sensor data regarding the physical activities of many different users and store the results.

In some instances, the server 412 may be provided with sensor data from test subjects performing known physical activities to form the basis for determining a given physical activity from the sensor data. For example, a user may record sensor data while jogging, and long that data with an entry “jogging for five minutes”. A number of such training samples produce a library of associations between sensor data and physical activity. The user may also choose to manually note a particular physical activity without providing sensor data simultaneously.

The server 412 may also form a user profile based on the estimated user physical activity pattern characterized by the sensor data. For example, if a user frequently bicycles during weekday mornings, the server 412 may add that user to a list of other users who also bicycle frequently during weekday mornings. Thus, similar user profiles may be compiled into a user group, even if the various users are strangers to each other. User groups may be defined for a given geographical area as well as user physical activity and timing, based on the sensor location data.

The server 412 may also associate the user profile with recommendations for products and/or services that may be relevant to a particular user or user group. That is, the sensors in the client machine 402 may control the recommendations that the server 412 may present to users of a networked publication and marketing system, for example. The previous purchases of the user and/or of members of a user group defined by its physical activity pattern are likely to be relevant to a user with a similar physical activity pattern. In one embodiment, the server 412 may be owned by a networked publication and marketing service, although this disclosure is not limited in that regard.

For example, suppose sensor data for particular user indicates this user does not sleep well, but instead does an unusual amount of tossing and turning, compared for example to the user's own history or to the histories of other users. This user may be associated with a group of other users who also share this physical activity pattern. The server 412 may then determine that products and services that were previously purchased by the group of other users might be of interest to the particular user. The relevant products or services may be related to sleeping, and may include, for example, pillows, sheets, sleeping pills, or late-night entertainment content. Thus, the combination of user physical activity patterns and previous purchases of the user and/or other users sharing similar physical activity patterns may define a market segment and corresponding products or services relevant to that market segment.

Similarly, a market segment of users may include bicyclists, and server 412 may provide them with bicycling related recommendations. The timing of the recommendations may correspond with the timing of sensor data indicating that a particular user is currently riding a bicycle. In another example, the timing of the recommendations may correspond with the timing of sensor data indicating that a particular user has just finished riding a bicycle. In either case, the recommendations may be for bicycling related goods and services, such as sports drinks, vitamins and other supplements, exercise equipment, nutrition-based diet suggestions, information regarding competitions, apparel, sports event tickets, gymnasium memberships, shoes, subscriptions to health newsletters or magazines, and so forth.

The server 412 may send the recommendations for relevant products or services to an application 414. The application 414 may be the same as application 410 that collected the sensor data, or it may be a separate application. Application 414 may be executing on client machine 402 or a different client machine, or it may be executing on a different platform, such as server 412. Application 414 may comprise an email tool or a web browser presenting a web page, for example, although this disclosure is not limited in this regard. The application 414 may display recommendations when it is first started, or when sensor data indicates a user has finished a given physical activity or arrived at a given location.

Application 414 may display a recommendations window 416, for example. Application 414 may also provide an icon or link to enable a user to readily take an action in response to the recommendation(s). For example, in icon 418, the user may be able to directly buy a suggested item from a list of suggested items. Application 414 may then transmit this user choice to a networked publication system or ecommerce system via instruction 420.

Many types of items may be of interest to a user, such as informational items (e.g., news articles, blogs, images, or multimedia content) and transactional items (e.g., items for sale or items wanted). Exemplary items may be goods that are purchased (e.g., a car, a pair of shoes, a movie ticket), an article (e.g., a news article, or a buying guide), a person's services (e.g., as a social contact, a professional contact, or a domain expert), or any other item that other users may be able to recommend. An item may also be a collection of items. Interactions with items may include viewing items, bidding on items, buying items, subscribing to items, and sharing the items on social networks. In some example embodiments, only a subset of the interactions may be considered. For example, only buying an item may be considered to be an interaction with the item.

Application 414 may also provide another icon or link 422 to enable a user to forward a recommendation to another user, such as a friend in a contact list. Application 414 may then transmit this user choice to the friend, for example, via an email application or via the networked publication system or ecommerce system via instruction 424. Thus, the recommendation may bring new users in as potential buyers who may be interested in the recommendation. In one embodiment, a user may both make a purchase and send a recommendation to a friend.

In summary, the recommendations of other users may be used to identify items and automatically provide one or more recommendations to the user requesting the recommendation. The provided recommendations may be presented in a user interface and operable to direct the user to the item. For example, the recommendation may be for a web site and presented as a hyperlink to the web site. As another example, the recommendation may be for a product available for purchase on another site and presented as a user interface element that, when clicked on, presents the user with the opportunity to purchase the item without leaving the original site.

The weight of the recommendations provided by other users may vary. For example, a user may set a weight for other users. To illustrate, a user may choose to give family members twice the weight of friends, or to give a user that typically gives bad advice a weight of zero, or even a negative weight. In some example embodiments, the weights for users may be automatically generated, and may be based on the previous interactions of the recommending user with items and the previous interactions of other users with items recommended by the recommending user.

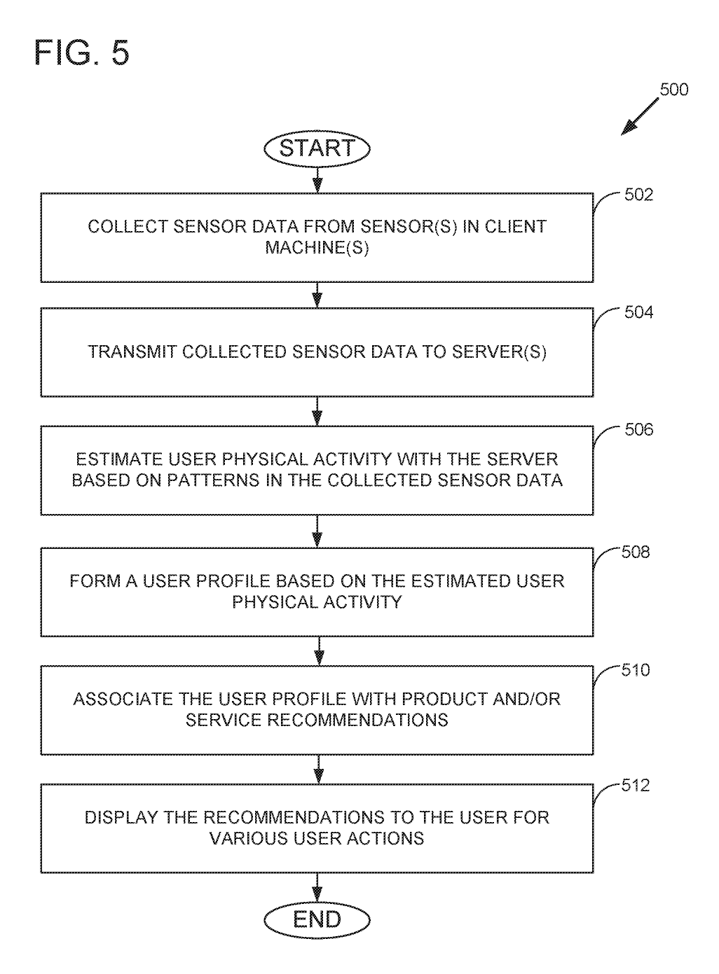

FIG. 5 is a flow chart 500 illustrating a method for processing sensor data collected by a client machine into relevant recommendations, in accordance with a disclosed embodiment. The method generally follows the operation of the system described above, and may be implemented as a set of program instructions that are stored in a computer-readable medium and executed by at least one processor. At 502, the method may collect sensor data from a number of sensors in a number of client machines or devices. At 504, the method may transmit the collected sensor data to a number of servers. Although this description is written in terms of the servers performing the analysis of sensor data and the generation of relevant recommendations, in some embodiments the client devices may perform these tasks in addition to or instead of the servers.

At 506, the method may estimate a user's physical activity using the server, based on patterns detected in the collected sensor data. At 508, the method may form a user profile based on the estimated user physical activity and, optionally, location. At 510, the method may associate the user profile with product or service recommendations. At 512, the method may display the recommendations to the user for various user actions, including making a purchase of a product or service, and forwarding the recommendations.

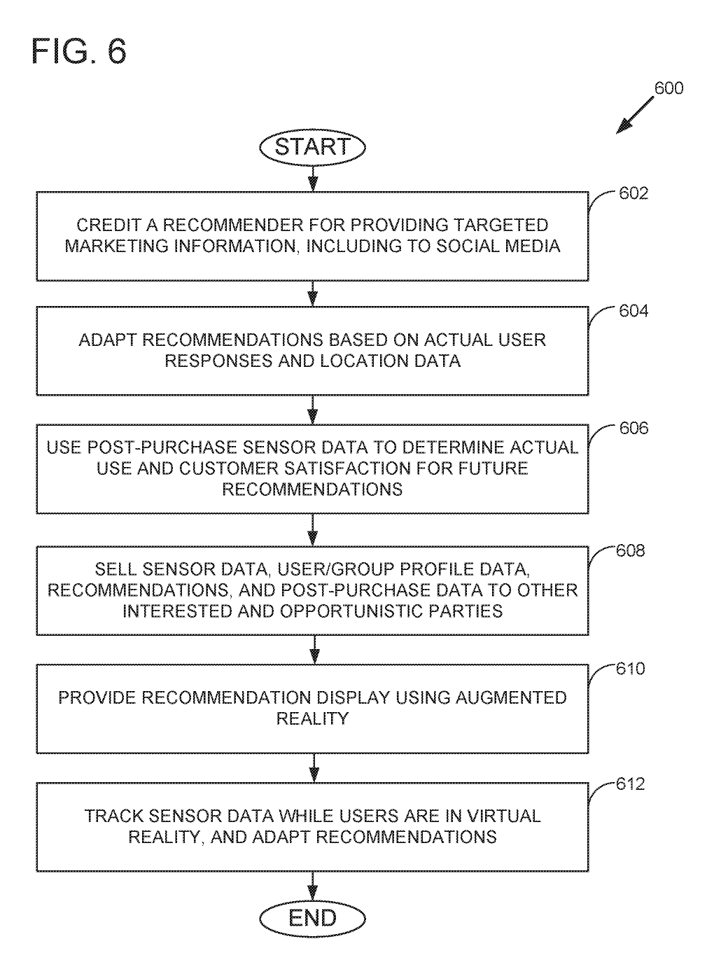

FIG. 6 is a flow chart 600 illustrating a method for processing sensor data collected by multiple client machines into relevant collaborative recommendations, according to an embodiment. This method may go beyond the method described above in several ways. For example, at 602, a networked publication system may credit an original shopper or customer for every suggestion made to another user. The original shopper or customer may acquire further credits when a suggestion recipient makes a purchase. Credits may, for example, include cash or discounts on future purchases. The end result is that those who provide targeted marketing information are rewarded.

Further, a user may have an account on a social network (e.g., Facebook®, Twitter®, Linkedln®, etc.) The user may have relationships with other users in the social network (e.g., family, friends, colleagues). The user may also have relationships with other entities in the social network (e.g., the user may be a member of a group, an employee of a company, etc.). The social network may be a social portion of a commerce site.

Users may post recommendations on various social media sites for multiple recipients, including those who are strangers to them. High activity users may therefore provide a great deal of data to potential future purchasers, and may become “leaders” of a given user group or market segment. In another embodiment, users may post numerous recommendations on the networked publication and marketing system, and be recognized and rewarded for their trusted expertise in that forum as well.

Additionally, at 604, the method may monitor the results of initial recommendation sets and use those results to iteratively refine future recommendations. That is, an adaptive market segmentation strategy may improve its correlation between collected sensor data and estimates of user physical activities, and its correlation between user physical activities and product recommendations. For example, frequent morning bicyclists may not respond well to recommendations for new bicycles, but they may consistently stop at a coffee shop near the end of a bicycle ride. Recommendations based on location data at the end of a round of user physical activity may therefore lead to improved sales and/or sharing of revised recommendations with other users with similar habits. Similarly, as user habits and user group habits change over time, their corresponding profiles may be adapted to keep track.

The method may, at 606, also go beyond product recommendations to provide actual sensor data indicating customer satisfaction. For example, if an insomniac follows a recommendation for a better pillow by buying one, the subsequently collected and perhaps ongoing sensor data that indicates the customer is indeed sleeping better may serve as a strong but silent endorsement of the product. Data indicating actual product or service effectiveness may therefore be used to further refine future recommendations. Similarly, the purchaser of recommended new shoes may increase the amount of running performed each week, which may lead to increased weight loss or other positive effects that may prove influential for a seller.

In general, if a user follows a recommendation when it is made, this may be interpreted as a very strong endorsement of the recommendation's suitability for the user. However, if a user follows a recommendation and subsequent data indicates the recommendation actually resolved an issue for the user, that may comprise an even stronger endorsement of the recommendation.

At 608, the method may further include that sellers may pay the server for such sensor data and/or recommendations. In one example, sellers may provide real-time discounts to buyers based on buyer location and the actual customer satisfaction data of others in the same user profile. Thus, if another insomniac is near a vendor of the improved pillows, that vendor may induce a purchase by providing a timely incentive to a potential buyer, along with persuasive actual use and satisfaction data from previous sales to other buyers.

Similarly, if a potential buyer is leaving a doctor's office, that customer may be particularly receptive to relevant recommendations. Further, insurance companies may be very interested in acquiring exercise related data on customers or aggregate customer groups. A seller may therefore pay more for attentive and receptive sales leads to which targeted recommendations may be provided. Other parties may pay more for post-sales sensor data showing the effectiveness of a recommendation. The server may therefore act as an information broker by purchasing sensor data from a collection service, so the server may make that data available for improved generation of recommendations and/or verification of satisfaction. Thus, all players in the chain of sensor data may participate and increase profits by better targeting recommendations to potential and/or actual buyers and maintaining sensor data flow.

At 610, the method may display recommendations on the client machine using augmented reality. Devices that allow an overlay of machine-generated information onto a view of the world are increasing in popularity. Such portable heads-up displays may run applications that provide recommendations and directions to a vendor for example for an interested buyer to follow. The location and orientation of a user may be provided by a UPS sensor and a gyroscope, for example, to assist with the overlay process.

Further, at 612, the method may perform all of the actions previously described to track what a user is doing when participating in a virtual reality scenario. For example, if a user likes to virtually visit tourist destinations “on foot” while in an immersive virtual environment, the user may be receptive to recommendations for walking shoes, tourist guides, and air travel to the actual tourist sites they are first virtually visiting. Sensor data may therefore note whether a user is using a virtual reality system when performing various physical activities, and adapt its recommendations accordingly.

### Modules, Components and Logic

Certain embodiments are described herein as including logic or a number of components, modules, or mechanisms. A component is a tangible unit capable of performing certain operations and may be configured or arranged in a certain manner. In example embodiments, one or more computer systems (e.g., a standalone, client or server computer system) or one or more components of a computer system (e.g., a processor or a group of processors) may be configured by software (e.g., an application or application portion) as a component that operates to perform certain operations as described herein.

In various embodiments, a component may be implemented mechanically or electronically. For example, a component may comprise dedicated circuitry or logic that is permanently configured (e.g., as a special-purpose processor) to perform certain operations. A component may also comprise programmable logic or circuitry (e.g., as encompassed within a general-purpose processor or other programmable processor) that is temporarily configured by software to perform certain operations. It will be appreciated that the decision to implement a component mechanically, in dedicated and permanently configured circuitry, or in temporarily configured circuitry (e.g., configured by software) may be driven by cost and time considerations.

Accordingly, the term “component” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which components are temporarily configured (e.g., programmed), each of the components need not be configured or instantiated at any one instance in time. For example, where the components comprise a general-purpose processor configured using software, the general-purpose processor may be configured as respective different components at different times. Software may accordingly configure a processor, for example, to constitute a particular component at one instance of time and to constitute a different component at a different instance of time.

Components can provide information to, and receive information from, other components. Accordingly, the described components may be regarded as being communicatively coupled. Where multiple of such components exist contemporaneously, communications may be achieved through signal transmission (e.g., over appropriate circuits and buses) that connect the components. In embodiments in which multiple components are configured or instantiated at different times, communications between such components may be achieved, for example, through the storage and retrieval of information in memory structures to which the multiple components have access. For example, one component may perform an operation, and store the output of that operation in a memory device to which it is communicatively coupled. A further component may then, at a later time, access the memory device to retrieve and process the stored output. Components may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or more processors or processor-implemented modules. The performance of some of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., APIs).

### Electronic Apparatus And System

Example embodiments may be implemented in digital electronic circuitry, or in computer hardware, firmware, software, or in combinations thereof. Example embodiments may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry, e.g., a field programmable gate array (FPGA) or an application-specific integrated circuit (ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware machine) and software architectures that may be deployed, in various example embodiments.

### Example Three-Tier Software Architecture

In some embodiments, the described methods may be implemented using one of a distributed or non-distributed software application designed under a Three-tier architecture paradigm. Under this paradigm, various parts of computer code (or software) that instantiate or configure components or modules may be categorized as belonging to one or more of these three tiers. Some embodiments may include a first tier as an interface (e.g., an interface tier). Further, a second tier may be a logic (or application) tier that performs application processing of data inputted through the interface level. The logic tier may communicate the results of such processing to the interface tier, and/or to a backend, or storage tier. The processing performed by the logic tier may relate to certain rules, or processes that govern the software as a whole. A third, storage tier, may be a persistent storage medium, or a non-persistent storage medium. In some cases, one or more of these tiers may be collapsed into another, resulting in a two-tier architecture, or even a one-tier architecture. For example, the interface and logic tiers may be consolidated, or the logic and storage tiers may be consolidated, as in the case of a software application with an embedded database. The three-tier architecture may be implemented using one technology, or, a variety of technologies. The example three-tier architecture, and the technologies through which it is implemented, may be realized on one or more computer systems operating, for example, as a standalone system, or organized in a server-client, peer-to-peer, distributed or some other suitable configuration. Further, these three tiers may be distributed between more than one computer systems as various components.

### Components

Example embodiments may include the above described tiers, and processes or operations about constituting these tiers may be implemented as components. Common to many of these components is the ability to generate, use, and manipulate data. The components, and the functionality associated with each, may form part of standalone, client, server, or peer computer systems. The various components may be implemented by a computer system on an as-needed basis. These components may include software written in an object-oriented computer language such that a component oriented, or object-oriented programming technique can be implemented using a Visual Component Library (VCL), Component Library for Cross Platform (CLX), Java Beans (JB), Java Enterprise Beans (EJB), Component Object Model (COM), Distributed Component Object Model (DCOM), or other suitable technique.

Software for these components may further enable communicative coupling to other components (e.g., via various APIs), and may be compiled into one complete server, client, and/or peer software application. Further, these APIs may be able to communicate through various distributed programming protocols as distributed computing components.

### Distributed Computing Components and Protocols

Some example embodiments may include remote procedure calls being used to implement one or more of the above described components across a distributed programming environment as distributed computing components. For example, an interface component (e.g., an interface tier) may form part of a first computer system that is remotely located from a second computer system containing a logic component (e.g., a logic tier). These first and second computer systems may be configured in a standalone, server-client, peer-to-peer, or some other suitable configuration. Software for the components may be written using the above described object-oriented programming techniques, and can be written in the same programming language, or a different programming language. Various protocols may be implemented to enable these various components to communicate regardless of the programming language used to write these components. For example, a component written in C++ may be able to communicate with another component written in the Java programming language through utilizing a distributed computing protocol such as a Common Object Request Broker Architecture (CORBA), a Simple Object Access Protocol (SOAP), or some other suitable protocol. Some embodiments may include the use of one or more of these protocols with the various protocols outlined in the Open Systems Interconnection (OSI) model, or Transmission Control Protocol/Internet Protocol (TCP/IP) protocol stack model for defining the protocols used by a network to transmit data.

### A System of Transmission Between a Server and Client

Example embodiments may use the Open Systems Interconnection (OSI) model or Transfer Control Protocol/Internet Protocol (TCP/IP) protocol stack model for defining the protocols used by a network to transmit data. In applying these models, a system of data transmission between a server and client, or between peer computer systems, may, for example, include five layers comprising: an application layer, a transport layer, a network layer, a data link layer, and a physical layer. In the case of software, for instantiating or configuring components, having a three-tier architecture, the various tiers (e.g., the interface, logic, and storage tiers) reside on the application layer of the TCP/IP protocol stack. In an example implementation using the TCP/IP protocol stack model, data from an application residing at the application layer is loaded into the data load field of a TCP segment residing at the transport layer. This TCP segment also contains port information for a recipient software application residing remotely. This TCP segment is loaded into the data load field of an IP datagram residing at the network layer. Next, this IP datagram is loaded into a frame residing at the data link layer. This frame is then encoded at the physical layer and the data transmitted over a network such as an internet, Local Area Network (LAN), Wide Area Network (WAN), or some other suitable network some cases, internet refers to a network of networks. These networks may use a variety of protocols for the exchange of data, including the aforementioned TCP/IP, and additionally Asynchronous Transfer Mode (ATM), Systems Network Architecture (SNA), Serial Digital Interface (SDI), or some other suitable protocol. These networks may be organized within a variety of topologies (e.g., a star topology), or structures.

Although an embodiment has been described with reference to specific example embodiments, it will be evident that various modifications and changes may be made to these embodiments without departing from the broader spirit and scope of the embodiment. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

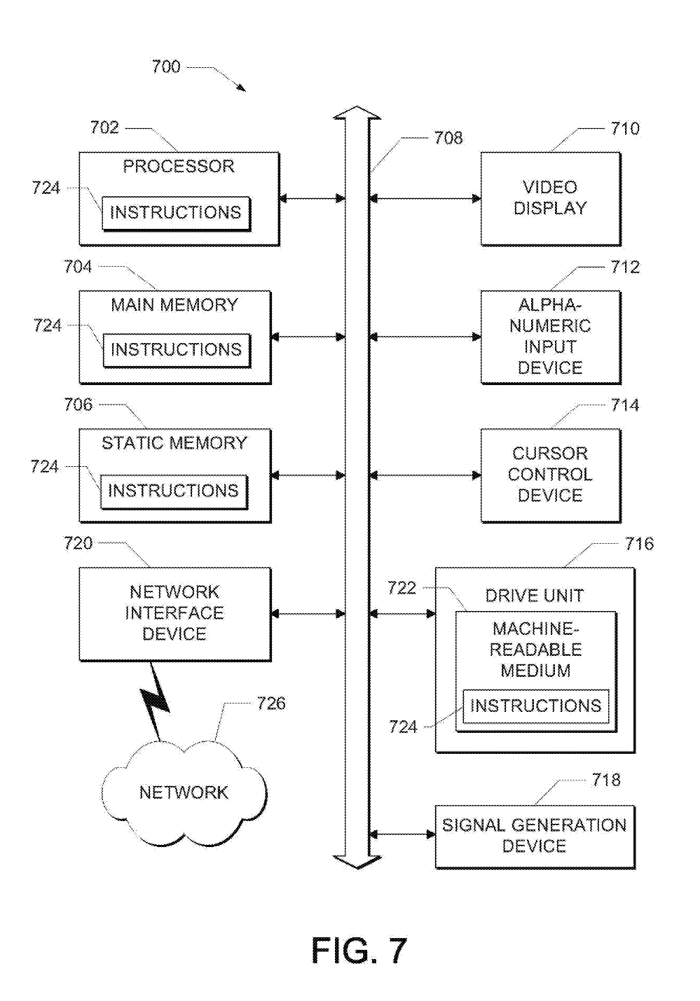

FIG. 7 shows a diagrammatic representation of a machine in the example form of a computer system 700 within which a set of instructions, for causing the machine to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 700 includes a processor 702 (e.g., CPU, a graphics processing unit (GPU), or both), a main memory 704, and a static memory 706, which communicate with each other via a bus 708. The computer system 700 may further include a video display unit 710 (e.g., a liquid crystal display (LCD) or a cathode ray tube (CRT)). The computer system 700 also includes an alphanumeric input device 712 (e.g., a keyboard), a cursor control device 714 (e.g., a mouse), a disk drive unit 716, a signal generation device 718 (e.g., a speaker), and a network interface device 720.

The disk drive unit 716 includes a machine-readable medium 722 on which is stored one or more sets of instructions (e.g., software 724) embodying any one or more of the methodologies or functions described herein. The software 724 may also reside, completely or at least partially, within the static memory 706, the main memory 704, and/or within the processor 702 during execution thereof by the computer system 700, the main memory 704 and the processor 702 also constituting machine-readable media.

The software 724 may further be transmitted or received over a network 726 via the network interface device 720.

While the machine-readable medium 722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies described herein. The term “machine-readable medium” shall accordingly be taken to include, but not be limited to, solid-state memories, and optical and magnetic media.

### Data Structures

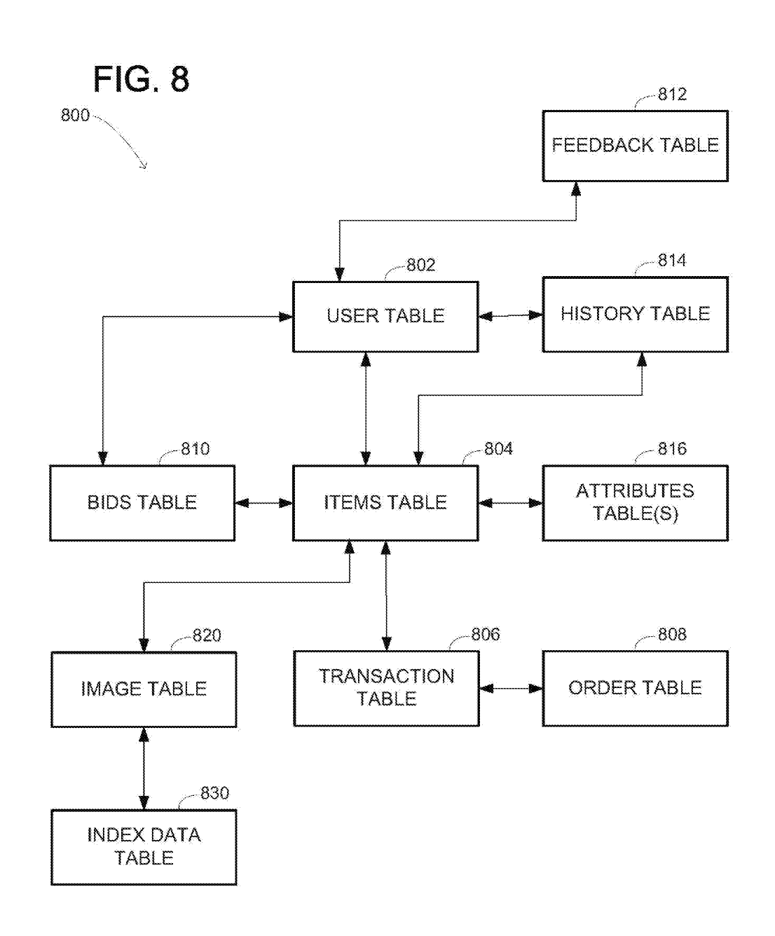

FIG. 8 is a high-level entity-relationship diagram of an example embodiment, illustrating various tables 800 that may be maintained within the databases 35 to 37, and that are utilized by and support the applications 30 and 32. A user table 802 contains a record for each registered user of the network-based marketplace platform 12, and may include identifier, address, and financial instrument information pertaining to each such registered user. A user may operate as a seller, a buyer, or both, within the network-based marketplace platform 12. In one example embodiment, a buyer may be a user that has accumulated value (e.g., commercial or proprietary currency), and is accordingly able to exchange the accumulated value for items that are offered for sale by the network-based marketplace platform 12. The user table 802 may also contain sensor data for a user.

The tables 800 also include an items table 804 in which are maintained item records for goods and services that are available to be, or have been, transacted via the network-based marketplace platform 12. Each item record within the items table 804 may furthermore be linked to one or more user records within the user table 802, so as to associate a seller and one or more actual or potential buyers with each item record.

The items table 804 may be connected to an image table 820, which contains images associated with the respective items or item listings in the items table 804. The image table 820 is in turn connected to an index data table 830, which contains index data as described in detail above.

A transaction table 806 contains a record for each transaction (e.g., a purchase or sale transaction) pertaining to items for which records exist within the items table 804. The transaction table 806 may also contain recommendations that have been provided to the user based on physical activity determined from sensor data and on products purchased by other similar users.

An order table 808 is populated with order records, with each order record being associated with an order. Each order, in turn, may correspond to one or more transactions for which records exist within the transaction table 806. The order table 808 may also contain indications of whether an order was based on a recommendation provided based on sensor data.

Bid records within a bids table 810 each relate to a bid received at the network-based marketplace platform 12 in connection with an auction-format listing supported by an auction application 32. A feedback table 812 is utilized by one or more reputation applications 50, in one example embodiment, to construct and maintain reputation information concerning users. A history table 814 maintains a history of transactions to which a user has been a party. One or more attributes tables 816 record attribute information pertaining to items for which records exist within the items table 804. Considering only a single example of such an attribute, the attributes tables 816 may indicate a currency attribute associated with a particular item, with the currency attribute identifying the currency of a price for the relevant item as specified by a seller.

Thus, a method and system to provide sensor based product recommendations in a network-based marketplace have been described. Although the present method and system have been described with reference to specific example embodiments, it will be evident that various modifications and changes may be made to these embodiments without departing from the broader spirit and scope of the application. Accordingly, the specification and drawings are to be regarded in an illustrative rather than a restrictive sense.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A method for generating an item recommendation, the method comprising:
collecting sensor data with a sensor client machine;
transferring the collected sensor data to a server;
estimating user physical activity based on collected sensor data patterns;
forming a user profile based on the estimated user physical activity;
associating the user profile with an item recommendation; and
displaying the item recommendation to a user.

2. The method of claim 1, wherein the item recommendation comprises at least one of a service recommendation and a product recommendation.

3. The method of claim 1, wherein the sensor data describes at least one of an acceleration, an orientation, a location, a proximity, an ambient environmental condition, biometric indicia, and user interactions with the client machine.

4. The method of claim 1, wherein the sensor data is updated one of continuously and periodically.

5. The method of claim 1, wherein the user physical activity comprises at least one of sleeping, walking, jogging, running, bicycling, driving, weight training, and remaining still.

6. The method of claim 1, wherein the associating comprises finding correlations with previous sales to at least one of the user and other users having similar user profiles.

7. The method of claim 1, further comprising at least one of purchasing an item based on the displayed recommendation and sharing the displayed recommendation with another user.

8. The method of claim 1, wherein the displayed recommendation includes a discount determined by the user profile.

9. The method of claim 1, further comprising at least one of the user and the server providing subsequent sensor data showing satisfaction with a purchased item.

10. The method of claim 1, wherein the collected sensor data describes user physical activity in virtual reality and the recommendation is displayed in virtual reality.

11. A non-transitory computer-readable storage medium having embedded therein a set of instructions which, when executed by one or more processors of a computer, causes the computer to execute the following operations for generating an item recommendation:
collecting sensor data with a sensor in a client machine;
transferring the collected sensor data to a server;
estimating user physical activity with the server based on collected sensor data patterns;
forming a user profile based on the estimated user physical activity;
associating the user profile with an item recommendation; and
displaying the item recommendation to a user.

12. The medium of claim 11, wherein the item recommendation comprises at least one of a service recommendation and a product recommendation.

13. The medium of claim 11, wherein the sensor data describes at least one of an acceleration, an orientation, a location, a proximity, an ambient environmental condition, biometric indicia, and user interactions with the client machine.

14. The medium of claim 11, wherein the user physical activity comprises at least one of sleeping, walking, jogging, running, bicycling, driving, weight training, and remaining still.

15. The medium of claim 11, wherein the associating comprises finding correlations with previous sales to at least one of the user and other users having similar user profiles.

16. The medium of claim 11, further comprising operations for at least one of purchasing an item based on the displayed recommendation and sharing the displayed recommendation with another user.

17. The medium of claim 11, wherein the displayed recommendation includes a discount determined by the user profile.

18. The medium of claim 11, further comprising operations for at least one of the user and the server providing subsequent sensor data showing satisfaction with a purchased item.

19. The medium of claim 11, wherein the collected sensor data describes user physical activity in virtual reality and the recommendation is displayed in virtual reality.

20. A system for generating an item recommendation, the system comprising:
at least one client machine that collects sensor data with at least one sensor and transfers the collected sensor data to a server that:
estimates user physical activity with the server based on patterns in the collected sensor data;
forms a user profile based on the estimated user physical activity;
associates the user profile with at least one item recommendation; and
a display that presents the item recommendation to the user.
